(19) United States
(12) Patent Application Publication
(10) Pub. No.: US 2021/0030583 A1
(43) Pub. Date: Feb. 4, 2021

(54) Anti-Snoring Multiple Height and Slope Pillow

(71) Applicant: Jose Manuel Chanfrau, IV, Miami Beach, FL (US)

(72) Inventor: Jose Manuel Chanfrau, IV, Miami Beach, FL (US)

(73) Assignee: Silentium, LLC, Miami Beach, FL (US)

(21) Appl. No.: 16/531,071

(22) Filed: Aug. 4, 2019

(51) Int. Cl. A61F 5/56; A47G 9/10

(52) U.S. Cl. CPC A61F 5/56; A47G 2009/1018; A47G 9/1072

(57) ABSTRACT

This invention relates to pillows, specifically to a pillow that provides support for a person's head, back in treating snoring and obstructive sleep apnea syndrome. This invention is constructed with firm polyurethane foam, ultimately allowing an adequate support of the head and neck with three different heights and angles to support the head and neck in what is generally described as a “Fowler's Position” where the person is sitting straight up or leaning slightly back. The invention achieves the Fowler's position by having the person lying on the bed in a supine position using the pillow which has angles to rest the head on steps that allow the head and neck to be position at heights from 3, 6 or 11.5 inches. The head will rest on a “step” that will vary from 40 degrees, 34 degrees or 11.3 degrees. The person using the pillow will determine the proper combination of pillow height and angle that works best for positioning the user's head for comfort and snoring reduction or elimination.

### BACKGROUND OF THE INVENTION

### 1. Technical Field of the Invention

This invention relates to pillows, specifically to a pillow that provides support for a person's head, back in treating snoring and obstructive sleep apnea syndrome (OSAS).

### 2. Summary of the Invention

This invention is constructed with firm polyurethane foam, ultimately allowing an adequate support of the head and neck with three different heights and angles to support the head and neck in what is generally described as a “Fowler's Position” where the person is sitting straight up or leaning slightly back. The “Fowler's Position” is generally preferred as an option in respiratory distress syndrome since it allows better chest expansion and improves breathing by facilitating oxygenation than a supine, recumbent or prone position.

### BRIEF DESCRIPTION OF THE DRAWINGS

FIG. 1 shows the general position of the person on one of the pillow's multiple steps and the pillow's dimensions and composition.

FIG. 2 shows a top view of the pillow that displays its multiple steps and the pillow's dimensions.

FIG. 3 shows a back view of the pillow that displays its dimensions and that it is flat in the back so that it is flush with a bed's headboard.

FIG. 4A shows the position of a person on the top “step” of the pillow at a 34-degree angle.

FIG. 4B shows the position of a person on the bottom “step” of the pillow at a 40-degree angle.

FIG. 5 shows a top view of the pillow that displays the slope of its multiple steps and the pillow's dimensions.

FIG. 6 shows a bottom view of the pillow that displays that it is flat on the bottom to be placed on top of a mattress.

### DETAILED DESCRIPTION OF THE DRAWINGS

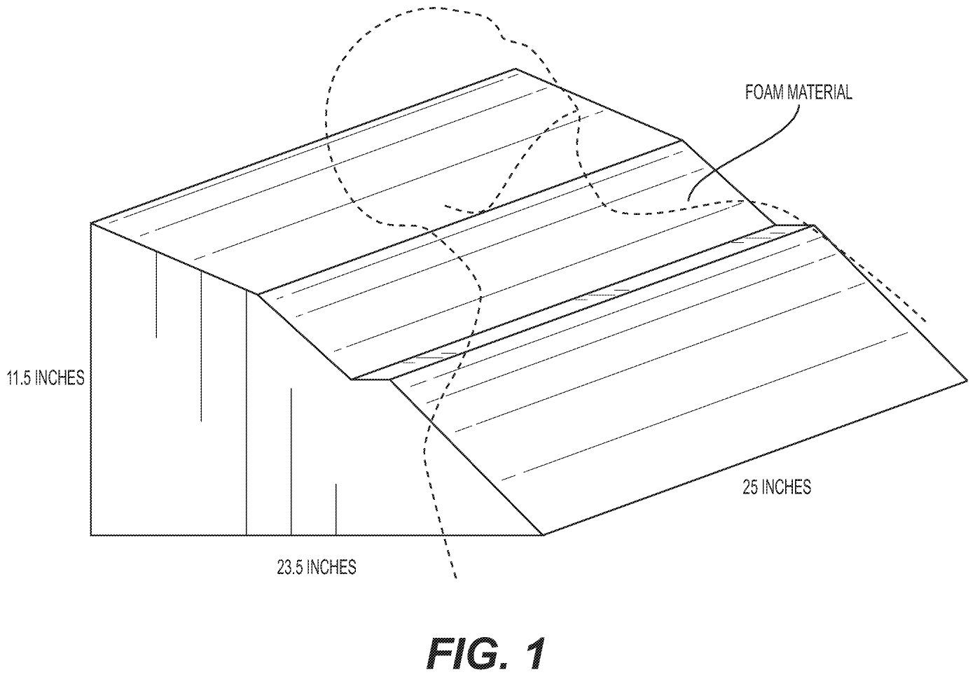

a. FIG. 1 shows the general position of the person on one of the pillow's multiple steps and the pillow's dimensions and composition. The person using the pillow is in what is generally described as a “Fowler's Position” where the person is sitting straight up or leaning slightly back. The “Fowler's Position” is generally preferred as an option in respiratory distress syndrome since it allows better chest expansion and improves breathing by facilitating oxygenation than a supine, recumbent or prone position.

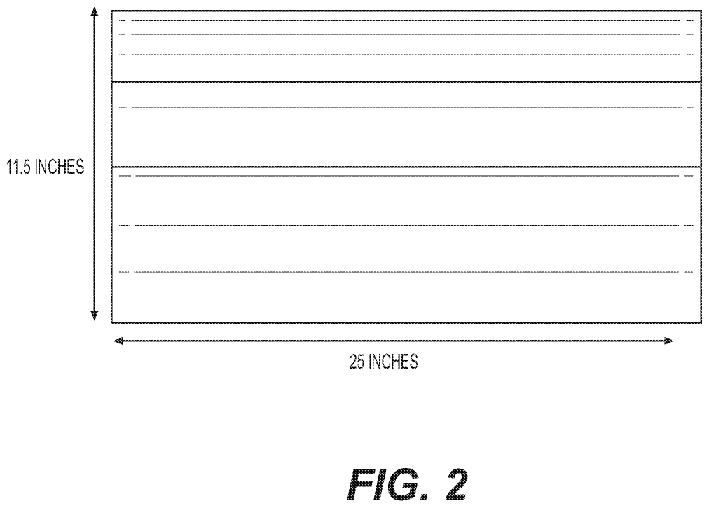

b. FIG. 2 shows a top view of the pillow that displays its multiple steps and the pillow's dimensions. The pillow which has angles to rest the head on steps that allow the head and neck to be position at heights from 3, 6 or 11.5 inches. The head will rest on a “step” that will vary from 40 degrees, 34 degrees or 11.3 degrees. The person using the pillow will determine the proper combination of pillow height and angle that works best for positioning the user's head for comfort and snoring reduction or elimination.

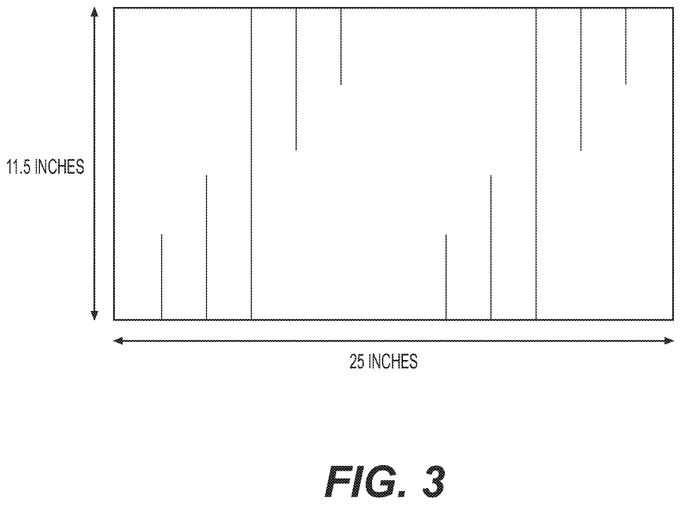

c. FIG. 3 shows a back view of the pillow that displays its dimensions and that it is flat in the back so that it is flush with a bed's headboard. Because the pillow is braced by the headboard it does not move and allows the user to maintain what is generally described as a “Fowler's Position” where the person is sitting straight up or leaning slightly back.

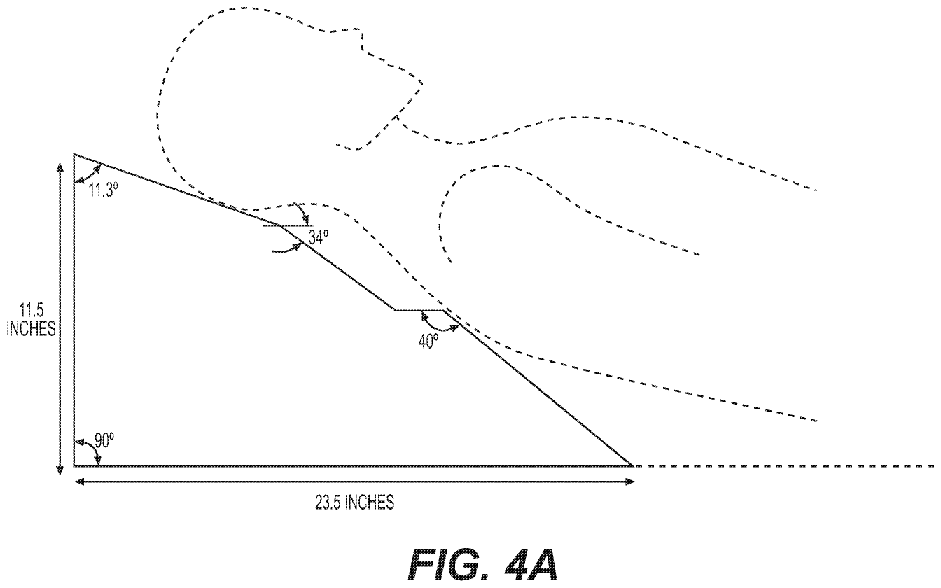

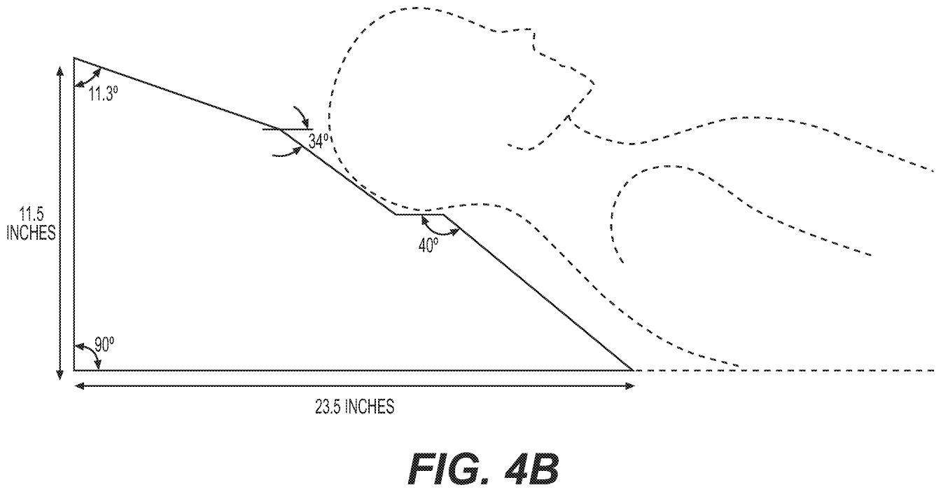

d. FIG. 4A shows the position of a person on the top “step” of the pillow at a 34-degree angle. The person lying on the bed is in a supine position using the pillow at its maximum height.

e. FIG. 4B shows the position of a person on the bottom “step” of the pillow at a 40-degree angle. The person lying on the bed is in a supine position using the pillow at its minimum height.

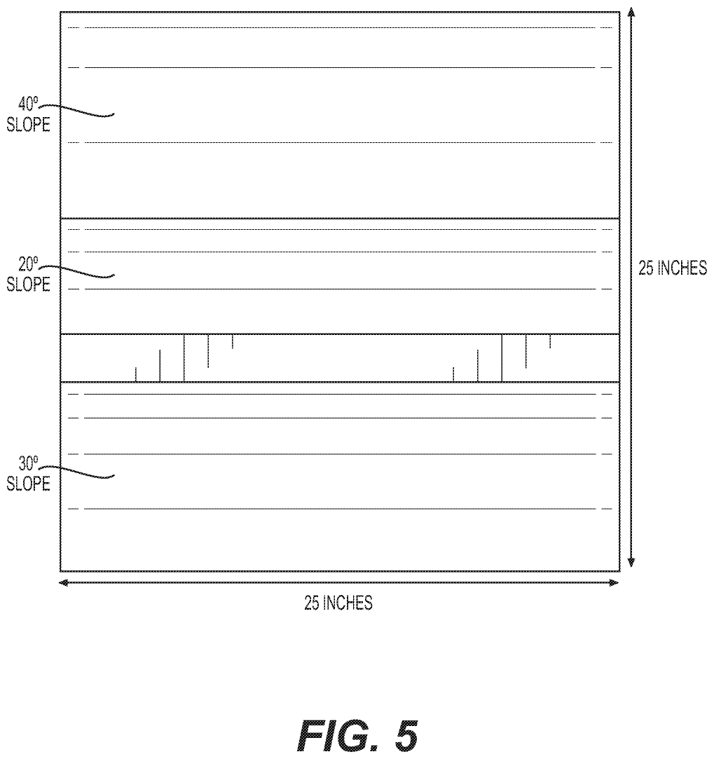

f. FIG. 5 shows a top view of the pillow that displays the slope of its multiple steps and the pillow's dimensions. The drawing shows the combination of angles to rest the head on steps that allow the head and neck to be position at heights from 3, 6 or 11.5 inches. The head will rest on a “step” that will vary from 40 degrees, 34 degrees or 11.3 degrees. The person using the pillow will determine the proper combination of pillow height and angle that works best for positioning the user's head for comfort and snoring reduction or elimination.

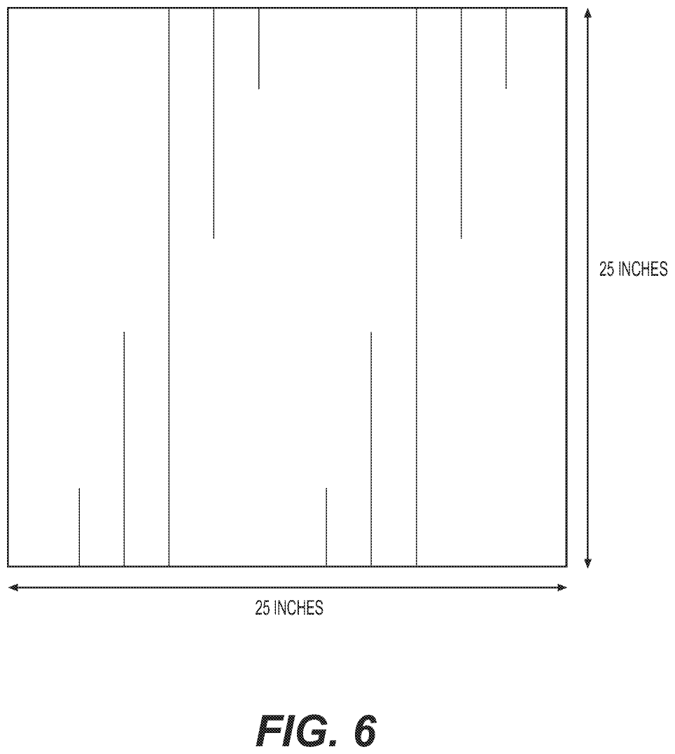

g. FIG. 6 shows a bottom view of the pillow that displays that it is 25 inches wide and flat on the bottom to be placed on top of a mattress. Because the bottom and the back are flat, the pillow is anchored by the wide amount of material on the bottom and the headboard.

What is claimed:

1. This invention relates to pillows, specifically to a pillow that provides support for a person's head, and back in treating snoring and obstructive sleep apnea syndrome (OSAS): OSAS is a widespread sleep disorder affecting 2-4% of the middle-aged population. Position therapy plays a role in treating snoring and OSAS to avoid supine sleeping and consequently reduce the severity of OSAS. Because the occurrence of pharyngeal collapse and snoring may be dependent on the head position, a head-positioning pillow designed to avoid supine sleep may serve as a simple and effective tool for reducing the severity of snoring in persons with uncomplicated mild-to-moderate positional OSAS.
Most snoring treatments involve use of continuous positive airway pressure (CPAP) devices or devices that restrict the wearer's movement such as vests, neck-worn vibration systems, and sleep position trainers that due to their discomfort have a low compliance to the use of such devices. The invention avoids the pit falls of CPAP and devices that restrict the wearer's movement by allowing a person to (1) to elevate the head and neck during supine sleeping and (2) to allow free lateral sleeping without restrictions.
This invention is constructed with firm polyurethane foam, ultimately allowing an adequate support of the head and neck with three different heights and angles to support the head and neck in what is generally described as a “Fowler's Position” where the person is sitting straight up or leaning slightly back. The “Fowler's Position” is generally preferred as an option in respiratory distress syndrome since it allows better chest expansion and improves breathing by facilitating oxygenation than a supine, recumbent or prone position.
The invention achieves the Fowler's position by having the person lying on the bed in a supine position using the pillow which has angles to rest the head on steps that allow the head and neck to be position at heights from 3, 6 or 11.5 inches. The head will rest on a “step” that will vary from 40 degrees, 34 degrees or 11.3 degrees. The person using the pillow will determine the proper combination of pillow height and angle that works best for positioning the user's head for comfort and snoring reduction or elimination.
